(19) United States
(12) Patent Application Publication
(10) Pub. No.: US 2024/0138875 A1
(43) Pub. Date: May 2, 2024

(54) ILLUMINATED SURGICAL CANNULA

(71) Applicant: LUMITEX, INC, Strongsville, OH (US)

(72) Inventors: Joseph DOMBROWSKI, Medina, OH (US); Sara TOICH, North Ridgeville, OH (US); Nicolette DIEHL, Brunswick, OH (US)

(73) Assignee: LUMITEX, INC, Strongsville, OH (US)

(21) Appl. No.: 18/547,774

(22) PCT Filed: Mar. 23, 2022
(86) PCT No.: PCT/US2022/021479

§ 371 Date: Aug. 24, 2023

Related U.S. Application Data
(60) Provisional application No. 63/166,010, filed on Mar. 25, 2021.

(51) Int. Cl. A61B 17/34; A61B 17/00; A61B 90/35

(52) U.S. Cl. CPC A61B 17/3421; A61B 90/35; A61B 2017/00907

(57) ABSTRACT

A surgical cannula is provided for illuminating a surgical cavity using an optically transparent walled passageway having a working channel. Light from a light source is received by the surgical cannula and is emitted by the walled passageway to illuminate the working channel, such that both tissues accessible via the working channel and tissues surrounding the walled passageway are illuminated.

### RELATED APPLICATIONS

This application claims the benefit of 63/166,010 filed on Mar. 25, 2021. Which is herein incorporated by reference in its entirety.

### TECHNICAL FIELD

The present disclosure relates generally to surgical cannula and more particularly to an illuminated surgical cannula.

### BACKGROUND

When performing surgery, there is often a tradeoff between tissue damage and accessing and visualizing a surgical location. For example, to more fully view tissues in a surgical location, surgeons may need to make larger incisions to create a larger surgical cavity, as well as use surgical retractors for holding open the surgical cavity.

### SUMMARY

The present disclosure provides a surgical cannula for illuminating a surgical cavity using an optically transparent walled passageway and a light source for illuminating the working channel of the walled passageway, such that tissues accessible via the working channel and tissues surrounding the walled passageway are illuminated.

While several features are described herein with respect to embodiments of the invention; features described with respect to a given embodiment also may be employed in connection with other embodiments. The following description and the annexed drawings set forth certain illustrative embodiments of the invention. These embodiments are indicative, however, of but a few of the various ways in which the principles of the invention may be employed. Other objects, advantages, and novel features according to aspects of the invention will become apparent from the following detailed description when considered in conjunction with the drawings.

### BRIEF DESCRIPTION OF THE DRAWINGS

The annexed drawings, which are not necessarily to scale, show various aspects of the invention in which similar reference numerals are used to indicate the same or similar parts in the various views.

FIG. 1 is a perspective view of an exemplary embodiment of a surgical cannula including a separate dilator and retainer.

FIG. 2 is a perspective view of the surgical cannula of FIG. 2 with the dilator inserted into the retainer.

FIG. 3A is a perspective view of an exemplary embodiment of the surgical cannula including a light source having multiple light emitters.

FIG. 3B is a cut away side view of the surgical cannula of FIG. 3A.

FIG. 4A is a side cut away view of an exemplary embodiment of the surgical cannula including a light source having multiple light emitters.

FIG. 4B is a top view of the surgical cannula of FIG. 4A.

FIG. 5 is a side cut away view of an exemplary embodiment of the surgical cannula illuminated by an external light source.

FIG. 6A is a side cut away view of an exemplary embodiment of the surgical cannula including a light source and a linear illuminator.

FIG. 6B is a top view of the surgical cannula of FIG. 6A.

FIG. 7 is a side view of an exemplary embodiment of the surgical cannula including a light source and a linear illuminator located on an outer surface of the retainer.

FIG. 8A is a side cut away view of an exemplary embodiment of the surgical cannula including a light source and a planar illuminator.

FIG. 8B is a side perspective view of the surgical cannula of FIG. 8A.

The present invention is described below in detail with reference to the drawings. In the drawings, each element with a reference number is similar to other elements with the same reference number independent of any letter designation following the reference number. In the text, a reference number with a specific letter designation following the reference number refers to the specific element with the number and letter designation and a reference number without a specific letter designation refers to all elements with the same reference number independent of any letter designation following the reference number in the drawings.

### DETAILED DESCRIPTION

In a general embodiment, the present disclosure provides a surgical cannula for illuminating a surgical cavity using an optically transparent walled passageway having a working channel. Light from a light source is received by the surgical cannula and is emitted by the walled passageway to illuminate the working channel, such that tissues accessible via the working channel and tissues surrounding the walled passageway are both illuminated.

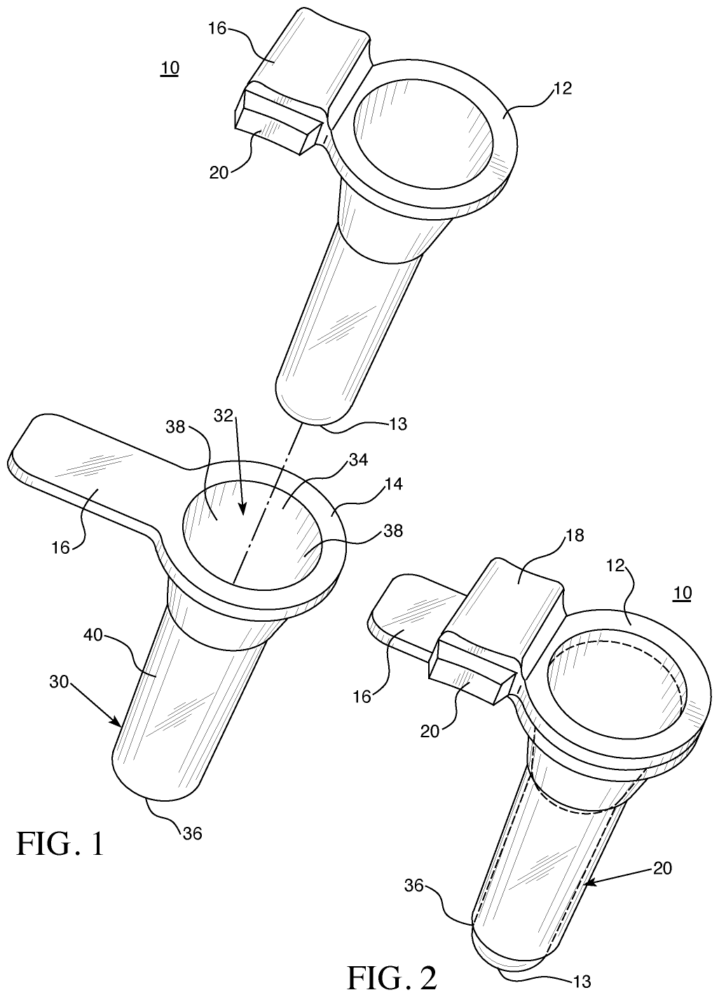

An exemplary embodiment of the surgical cannula 10 (also referred to as a surgical retractor) is shown in FIGS. 1 and 2. In this embodiment, the surgical cannula 10 includes a dilator 12 having a closed tip 13 and a retainer 14 having an open tip 15. In this embodiment, the dilator 12 pushes soft tissue of the brain out of the way as the retainer 14 is advanced into the brain. That is, the dilator 12 may be received within an interior working channel of the retainer 14, such that the closed tip 13 of the dilator 12 extends through and past the open tip 15 of the walled passageway 30 as shown in FIG. 2.

The dilator 12 and retainer 14 may be releasably attached via a clip 16. For example, the clip 16 may attach to a tab 18 of the retainer 14. The dilator 12 may then be removed while the retainer 14 remains in place to retain tissue out of the way and provide a working area for a surgeon. For example, a button 20 of the clip 16 may be depressed to disengage the clip 16 from the tab 18 so that the dilator 14 may be removed to open the working channel 32.

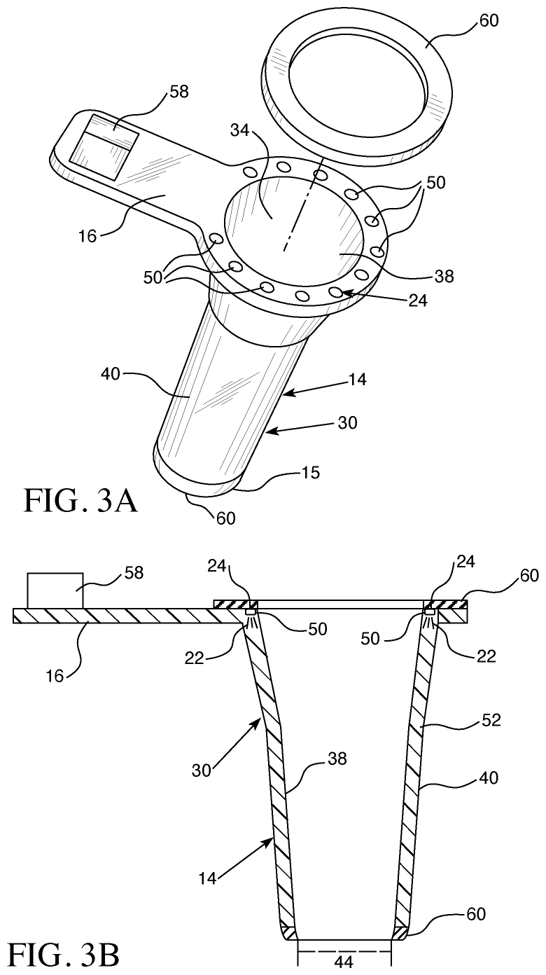

Turning to FIGS. 3A and 3B, an exemplary embodiment is shown of the surgical cannula 10 for illuminating a surgical cavity using light 22 emitted by a light source 24. The surgical cannula 10 includes an optically transparent walled passageway 30 having an interior working channel 32, an entrance 34, an open tip 15, an inner surface 38, an outer surface 40, and light-extracting features 42. The walled passageway 30 receives the light 22 emitted by the light source 24 and transports the received light 22 via total internal reflection towards the open tip 15 of the walled passageway 30. The light-extracting features 42 extract the transported light 22 from the walled passageway 30, such that the extracted light is emitted from the inner surface 38 of the walled passageway 30 into the working channel 32 and illuminates an area 44 bounded by the open tip 15 the walled passageway 30 (also referred to as a working area).

The light-extracting features may include both inward extracting structures and outward extracting structures. The inward extracting structures extract the transported light, such that the extracted light illuminates the area 44 bounded by the open tip 15 of the walled passageway 30. The outward extracting structures extract the transported light, such that the extracted light is emitted from the external surface 40 of the walled passageway 30. In this way, the outward extracting structures may be used to illuminate the environment (e.g., tissues) surrounding the walled passageway 30.

The light source 24 may include multiple light emitters 50 configured to emit the light 22. For example, as shown in FIG. 3A, each of the light emitters 50 may be positioned along the entrance 34 of the walled passageway 30, such that the light 22 emitted by each of the light emitters 50 is transported towards the open tip 15 of the walled passageway 30.

As described above, the walled passageway 30 is optically transparent. That is, the walls 40 of the walled passageway 30 allow at least a portion of the visible spectrum of light to pass through. For example, the walls 40 may be predominantly (e.g., at least 50%) clear. In one example, the walls 40 attenuate at most 10%, at most 20%, or at most 30% of the visible spectrum of light. The walled passageway 30 may be made of any suitable optically transparent material such as glass and/or plastic. In one embodiment, the walled passageway 30 is configured to be sterilized before use.

Each of the light emitters 50 may be enclosed within walls 52 of the walled passageway 30. For example, the light emitters 50 may be positioned within recesses in the walls 52 and the light emitters 50 may then be encapsulated within the recesses.

In one embodiment, the surgical cannula 10 includes a tab 18 integrally formed with the walled passageway 30. The tab 18 may be optically connected to the walled passageway 30 and the light source 24, such that the light 22 emitted by the light source 24 is received by the tab 18 and is transferred by total internal reflection from the tab 18 into the walled passageway 30.

the tab 18 may be any suitable structure mechanically attached to the walled passageway 30. For example, the tab 18 may be integrally or monolithically formed without the walled passageway 30.

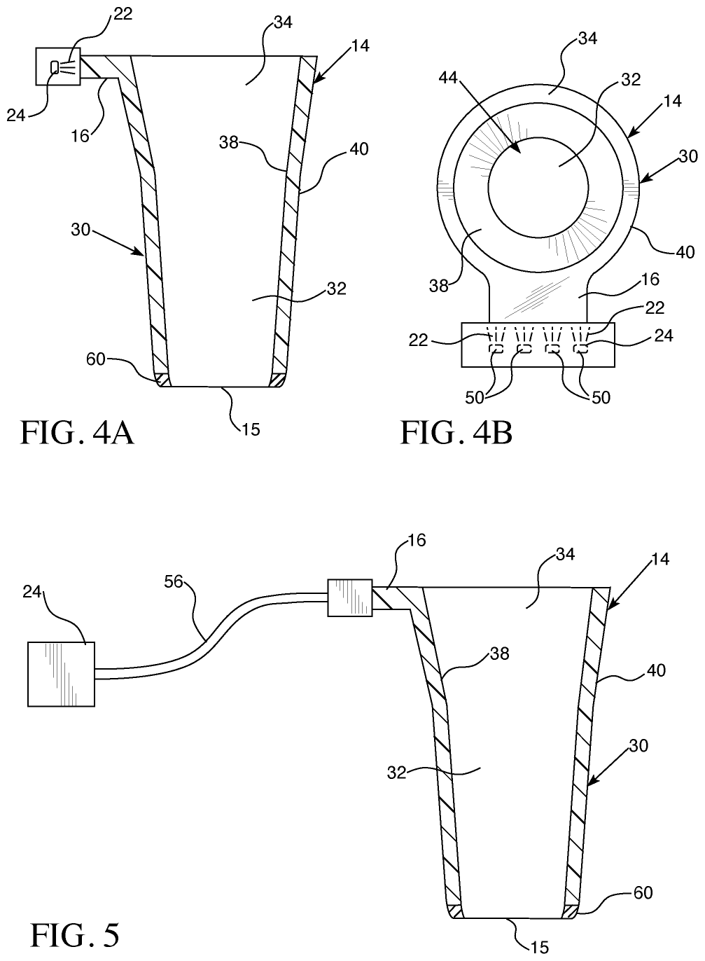

In the embodiment shown in FIGS. 4A and 4B, the surgical cannula 10 includes the light source 24 and the light source is mechanically supported by the tab 18, such that the light emitted 22 by the light source 24 is received by the tab 18. The light 22 received by the tab 18 may be transmitted from the tab 18 to the walls 40 of the walled passageway 30 and then within the walls towards the tip 15 of the walled passageway 30.

In the embodiment shown in FIG. 5, the light source 24 is not supported by the tab 18, but instead the light source 24 is optically connected to the tab 18 via a light guide 56 configured to transfer the light emitted by the light source to the tab 18. For example, the light source 24 may be an external light source optically connected to the tab 18 via an optical fiber.

In the embodiments shown in FIGS. 3A, 3B, 4A, 4B, and 5, the walled passageway 30 acts as a light guide for transporting the received light 22 from the light source towards the open tip 15. The light-extracting features extract the light being transmitted by the walls 40, such that the extracted light illuminates the area 44 bounded by the open tip 15 and/or tissues adjacent the outer surface 40 of the walled passageway 30 as described above.

The light extracting properties of the light-extracting features may vary along the walled passageway 30, such that illumination inside and outside of the working channel 32 appears uniform. For example, illumination of the area 44 bounded by the open tip 15 may vary in brightness by less than 40%, less than 30%, or less than 20%. Similarly, the illumination of the environment adjacent the outer wall 40 may vary in brightness by less than 40%, less than 30%, or less than 20%.

The light-extracting features may be configured to extract at least 60% of the light received by the walled passage 30, such that at most 40% of the received light is emitted from the open tip 15 of the walled passage 30. That is, the light-extracting features are configured to extract most of the light from the walled passageway 30 before the light reaches the open tip 15.

The light-extracting features may be used to control the uniformly of illumination provided by the surgical cannula 10. The light-extracting features may be any suitable structure for extracting light from a structure (such as a light guide) (e.g., to target a specific light output distribution). For example, the light-extracting features may include at least one of surface aberrations, micro-lenses, reflective spots, partial reflective planes, or diffraction gratings. Alternatively or additionally, a diffuser sheet or a 2-D lensing sheet may be (1) placed on an emission surface. In one embodiment, the surface aberrations include at least one of a contour of the surface, surface depositions, or surface etchings.

As shown in FIGS. 3A and 3B, the surgical cannula 10 may include a blocking agent 60 configured to optically attenuate light. For example, the open tip 15 may include the blocking agent 60, such that light emitted from the open tip 15 of the walled passageway 30 is reduced. Similarly, the entrance 34 may include the blocking agent 60, such that light emitted from the entrance 34 of the walled passageway 30 is reduced.

The blocking agent 60 may be any suitable material for attenuating light. For example, as shown in FIG. 3A, the blocking agent 60 may be material added to the walled passageway (such as dark plastic). As another example, the blocking agent 60 may be coloring added or applied to the walled passageway (such as dark paint or dye).

In one embodiment, the surgical cannula 10 also includes a power source 58 electrically connected to the light source 24. In addition to supporting the light source 24, the tab 18 and/or walled passageway 30 may also mechanically support the power source 58. The power source 58 may be any suitable source of electrical energy (e.g., a battery) for supplying electrical power to the light source 24.

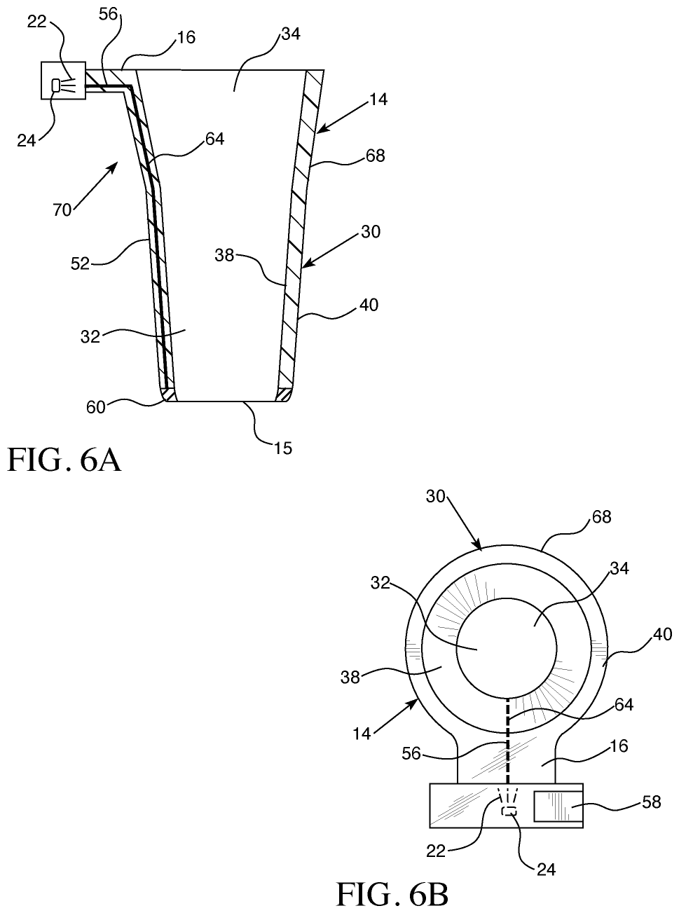

In the embodiment shown in FIGS. 6A and 6B, the surgical cannula 10 includes a light guide 56 and an illuminator 64. The light guide 56 is configured to receive the light 22 emitted by the light source 24 and to transmit the received light via total internal reflection to the illuminator 64. The illuminator 64 is configured to receive the light transmitted by the light guide 56 and to emit the light, such that the light is emitted from the inner surface 38 of the walled passageway 30 into the working channel 32 and illuminates the area 44 bounded by the open tip 15 the walled passageway 30.

In one embodiment, the illuminator 64 is positioned on one side of the walled passageway 30. The illuminator 64 may include light-extracting features for emitting light towards and away from the working channel 32. The light directed away from the working channel 32 may illuminate tissues outside of and on the same side of the walled passageway as the illuminator 64 (i.e., so that these tissues are visible to a surgeon looking through the walled passageway 30). The light directed towards the working channel 32 may be used to illuminate the area 44 bound by the open tip 15. Additionally, the light directed towards the working channel 32 may pass through both the working channel 32 and an opposite portion 68 of the walled passageway 30 located opposite the illuminator 64. In this way, any tissues located on an opposite side of the walled passageway 30 are also illuminated.

Embodiments of the surgical cannula 10 including the illuminator 64 may not include light-extracting features in the walled passageway 30. Instead, the light-extracting features may be present in the illuminator 64. Although, in some embodiments the surgical cannula 10 includes light-extracting features in both the illuminator 64 and the walled passageway 30.

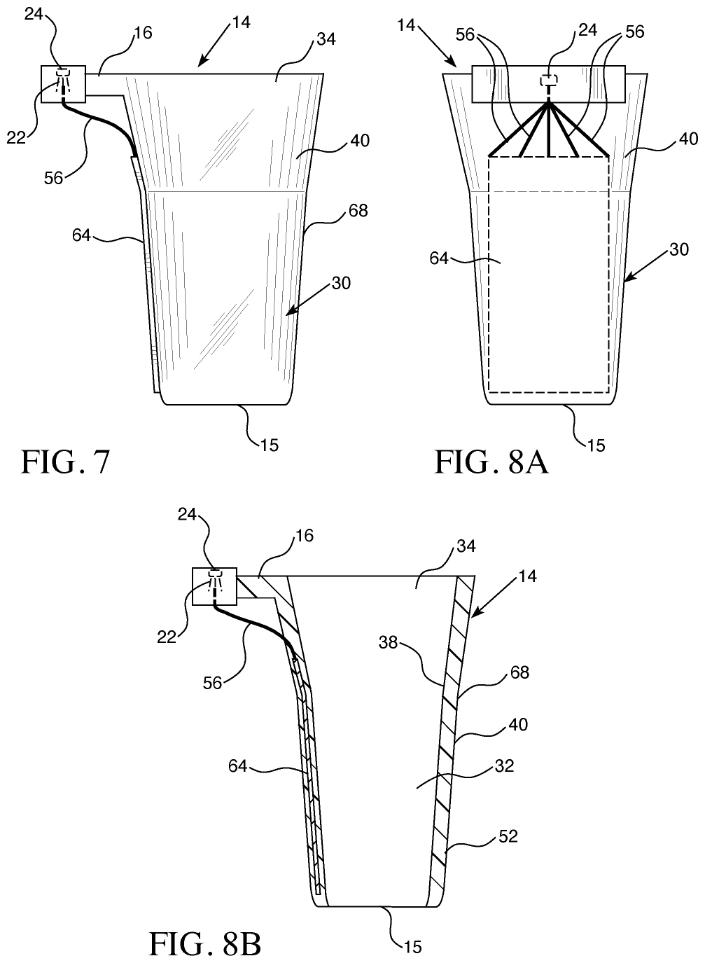

As shown in FIG. 6A, the illuminator 64 may be encapsulated within the walls 52 of the walled passageway 30, such that the illuminator is segregated from an external environment 70. For example, the illuminator 64 may be inserted into a channel in the wall 52 of the walled passageway 30. In the embodiment shown in FIG. 7, the illuminator 64 is attached to the outer surface 40 of the walled passageway 30. For example, the illuminator 64 may be adhered to the outer surface 40. In one embodiment, a coating may also be applied to the illuminator 64, such that the illuminator 64 is separated from and does not come in contact with the external environment 70.

As shown in FIGS. 6A, 6B, and 7, the illuminator 64 may be a linear light guide including light-extracting features configured to extract light from the linear light guide. For example, the illuminator 64 may be a fiber optic (e.g., monofilament fiber optic). Alternatively, as shown in FIGS. 8A and 8B, the illuminator 64 may be a planar light guide including light-extracting features configured to extract light from the planar light guide.

In one embodiment, the light source 24 may be attached to a clip for attaching to the tab 18. The clip may be used to maintain a position of the light source 24 relative to the tab 18 and/or a light guide 56. In another embodiment, the light guide 56 is attached to a clip that is used to optically connect an external light source to the tab 18. For example, the clip may have a circular or semicircular shape that attaches to the entrance 34 of the walled passageway 30.

The light source 24 (and light emitters 50) may be any suitable structure for emitting electromagnetic radiation. For example, the light source 24 may include one or more light emitting diodes (LEDs), organic LEDs (OLEDs), micro-LEDs, laser diodes, mini-LED, quantum dot (QD)-conversion, phosphor conversion, excimer lamps, multi-photon combination, or SLM wavefront manipulation.

The light 22 may include any suitable wavelengths of light. In addition to supplying light to improve visibility of a surgical cavity, the light source 24 may also include light having wavelengths useful for disinfecting the surgical cavity or for performing photobiomodulation. For example, the light 22 may include wavelengths (e.g., 600-1200 nm) configured to stimulate wound healing. As an example, the light source 24 may include multiple light emitters. One or more of the light emitters may emit the white light for visibility while other light emitter(s) emit photobiomodulation light.

In one embodiment, the surgical cannula 10 is used to illuminate a cranial surgical cavity or central nervous system surgical procedure. The surgical cannula 10 may be single use or sterilizable (i.e., multi-use).

All ranges and ratio limits disclosed in the specification and claims may be combined in any manner. Unless specifically stated otherwise, references to “a,” “an,” and/or “the” may include one or more than one, and that reference to an item in the singular may also include the item in the plural.

Although the invention has been shown and described with respect to a certain embodiment or embodiments, equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

1. A surgical cannula for illuminating a surgical cavity using light emitted by a light source, the surgical cannula comprising:
an optically transparent walled passageway having an interior working channel, an entrance, an open tip, an inner surface, an outer surface, and light-extracting features; and
multiple light emitters comprising the light source and configured to emit the light;
wherein each of the light emitters is positioned along the entrance of the walled passageway, such that the light emitted by each of the light emitters is transported via total internal reflection towards the open tip of the walled passageway;
wherein the walled passageway is configured to receive the light emitted by the light source and to transport the received light via total internal reflection towards the open tip of the walled passageway; and
wherein the light-extracting features are configured to extract the transported light from the walled passageway, such that the extracted light is emitted from the inner surface of the walled passageway into the working channel and illuminates an area bounded by the open tip the walled passageway.

2. The surgical cannula of claim 1, wherein the light-extracting features include:
inward extracting structures configured to extract the transported light, such that the extracted light illuminates the area bounded by the open tip of the walled passageway; and
outward extracting structures configured to extract the transported light, such that the extracted light is emitted from the outer surface of the walled passageway.

3-6. (canceled)

7. The surgical cannula of claim 1, wherein each of the light emitters is enclosed within walls of the walled passageway.

8. The surgical cannula of claim 1, further comprising a dilator having a closed tip, wherein:
the dilator is configured to be received within the interior working channel, such that the closed tip of the dilator extends through and past the open tip of the walled passageway.

9. The surgical cannula of claim 1, wherein light extracting properties of the light-extracting features varies along the walled passageway, such that illumination inside and outside of the working channel appears uniform.

10. The surgical cannula of claim 1, further comprising a power source mechanically supported by the walled passageway.

11. The surgical cannula of claim 1, wherein the light-extracting features extract at least 60% of the light received by the walled passage, such that at most 40% of the received light is emitted from the open tip of the walled passage.

12. The surgical cannula of claim 1, wherein at least one of:
the open tip includes a blocking agent configured to optically attenuate the transported light, such that light emitted from the open tip of the walled passageway is reduced; or
the entrance includes the blocking agent configured to optically attenuate the light emitted by the light source, such that light emitted from the entrance of the walled passageway is reduced.

13-20. (canceled)
